(19) United States
(12) Patent Application Publication
(10) Pub. No.: US 2023/0364025 A1
(43) Pub. Date: Nov. 16, 2023

(54) PROTEIN-RELEASE SYSTEM FOR SUSTAINED RELEASE OF PROTEINS

(71) Applicants: DANIELA FERNANDA ISAACS-BERNAL, ETOBICOKE (CA); MOLLY SHOICHET, TORONTO (CA); THE GOVERNING COUNCIL OF THE UNIVERSITY OF TORONTO, Toronto (CA)

(72) Inventors: DANIELA FERNANDA ISAACS-BERNAL, ETOBICOKE (CA); MOLLY SHOICHET, TORONTO (CA)

(21) Appl. No.: 18/143,649

(22) Filed: May 5, 2023

Related U.S. Application Data
(60) Provisional application No. 63/341,174, filed on May 12, 2022.

(51) Int. Cl. A61K 9/51; A61K 9/00; A61K 47/69; A61K 47/32

(52) U.S. Cl. CPC A61K 9/5153; A61K 9/0024; A61K 47/6903; A61K 47/6951; A61K 47/32

(57) ABSTRACT

The present disclosure provides a protein-release system for sustained release of proteins. The system includes a hydrogel comprising a polymeric network, proteins having a portion corresponding to a portion of a fragment crystallization (Fc) constant region of an antibody, and peptidic ligands covalently coupled to the polymeric network, the peptidic ligands comprising an amino acid sequence having a binding affinity to the portion of the Fc constant region, each protein being reversibly bound by affinity to the peptidic ligands.

### FIELD

The present disclosure relates to a protein-release system and method for sustained release of proteins, and more particularly this disclosure relates to a protein-release system for sustained release of antibodies from hydrogels.

### BACKGROUND

The clinical approval of new protein-based therapeutics for the treatment of various diseases has led to an increase in its market value (Lu et al., 2020). As of May 2021, approximately 100 therapeutic monoclonal antibodies have been approved by the United States Food and Drug Administration (US FDA) for treating cancer, neurological disorders, autoimmune and infectious diseases (Mullard, 2021). Despite this high growth potential, many protein-based therapeutics require repetitive dosing to achieve a long-term therapeutic benefit. For example, bevacizumab, an antibody against vascular endothelial growth factor (VEGF), is typically administered intravitreally once every 4-6 weeks to treat patients suffering from wet age-related macular degeneration (AMD) (Ba et al., 2015; Meyer & Holz, 2011; Wykoff et al., 2018). This highly invasive strategy works, but every injection comes with the risk of infection and is greatly undesirable for patients. Sustained release strategies for protein therapeutics aim to overcome these hurdles by maintaining therapeutic concentrations while using fewer injections, which would significantly benefit patient compliance and effectiveness of the treatment.

Affinity controlled release is a compelling strategy for the delivery of protein therapeutics because it obviates the need for protein encapsulation, which typically results in protein denaturation and minimal loading (Hettiaratchi & Shoichet, 2019). Affinity-controlled release utilizes non-covalent interactions between a protein of interest and an affinity ligand immobilized within a delivery vehicle. Modification of hydrogel matrices with affinity binders can be leveraged to achieve tunable protein release via manipulation of the protein-binding partner interactions, governed by the equilibrium dissociation constant (K_{D}) (Hettiaratchi et al., 2020).

Some of the present inventors have previously designed two affinity release strategies, one based on the affinity between Src homology 3 (SH3) chimera proteins and SH3-binding peptides (Delplace et al., 2020; Pakulska et al., 2017; Vulic & Shoichet, 2011), and the other based on the electrostatic interaction between negatively charged nanoparticles and positively-charged proteins (Pakulska et al. 2016).

Modified hydrogel matrices with SH3 peptidic ligands have been successful at controlling and tunning the release of several proteins expressed as fusion proteins with SH3 domains such as SH3-fibroblast growth factor (SH3-FGF2), SH3-insulin growth factor (SH3-IGF-1), SH3-ciliary neurotrophic factor (SH3-CNTF), and SH3-chondroitinase ABC (SH3-ChABC). However, while this strategy is highly attractive, it requires that bioactive proteins be expressed as SH3-fusion proteins. In order for this approach to work for antibodies, the molecular structure of antibodies of interest would require modification with an SH3 domain.

The electrostatic-mediated controlled release system modulates release by adsorption of positively-charged proteins to negatively-charged polymeric nanoparticles; however, some proteins will denature upon adsorption and others may not be sufficiently electropositive for their release to be controlled from the negatively-charged nanoparticles.

Another strategy that has been used to immobilize antibodies is through the interaction of protein G and the Fc region of antibodies. This affinity interaction is so strong that the antibody is not sufficiently released, making the protein G-Fc system undesirable for protein release strategies.

### SUMMARY

Herein, there is disclosed a sustained release strategy based on affinity interactions between proteins containing the Fragment Crystallizable domain (Fc) of antibodies and Fc-peptide ligands.

Thus, the present disclosure provides a protein-release system for sustained release of proteins, where the proteins having a portion corresponding to all or part of a fragment crystallization (Fc) constant region of an antibody. The protein-release system includes a hydrogel which comprises a polymeric network with peptidic ligands covalently coupled to the polymeric network. The peptidic ligands are selected to reversibly bind, by affinity, to the fragment crystallizable (Fc) constant region of the antibody Fc constant region of the proteins, so that they comprise an amino acid sequence having a binding affinity to the portion of the Fc constant region.

The present disclosure also provides a method of producing a physiologically acceptable protein-release system for sustained release of proteins, the proteins having a portion corresponding to a portion of a Fc constant region of an antibody. The method comprises selecting peptidic ligands known to have a binding affinity with the Fc constant region, covalently immobilizing the peptidic ligands to precursors of a polymeric network, mixing an aqueous solution of the polymeric network precursors, having the immobilized peptidic ligands, with the proteins and forming a hydrogel loaded with the proteins for sustained release.

The binding affinity may be characterized by a dissociation constant between the protein constant region and the peptidic ligand greater than about 10⁻¹⁰ M, and maybe in a range from about 10⁻⁴ to about 10⁻¹⁰ M.

More particularly the dissociation constant may be in a range from about 10⁻⁴ to about 10⁻⁹ M.

The peptide sequence has an amino acid sequence selected to give a preselected dissociation constant to give a selected release rate of the protein.

The polymeric network comprises naturally-derived polymer, synthetic polymer or a combination thereof, and the naturally-derived polymer, synthetic polymer or combination thereof is selected from the group consisting of poly(saccharides), hyaluronan or derivatives thereof, cellulose or derivatives thereof, amylose, amylopectin, collagen, gelatin, methylcellulose or derivatives thereof, alginate, chitosan, agarose, dextran, dextrin, oligomers, polyacrylates and derivatives thereof, polyacrylamide, poly(vinyl alcohol), poly(vinyl chlorides), poly(vinyl imidazoles), polyesters, poly(vinyl sulfides), poly(hydroxyl esters), ethylene-vinyl-acetates, poly(ethylene oxides), poly(propylene oxides), poloxamers, polysorbates, poly(propylene), polyurethanes, polystyrenes, poly(lactide), poly(glycolide), copolymers of lactide and glycolide, copolymers of lactide and poly(ethylene glycol), poly(ethylene glycol), copolymers of lactic acid and lactone, poly(peptides), polycaprolactone, polycarbonates, conjugates of PEG with poly(peptides), polyhydroxybutyrate, polyorthoesters, poly(propylene fumarate).

The polymeric network may be a click-crosslinked polymeric network.

The polymeric network may be a polymeric network of tetrazine-modified hyaluronan-aldehyde and/or hyaluronan-ketone/poly(ethylene glycol)-oxyamine.

The polymeric network may be a click-crosslinked polymeric network. Particularly, the polymeric network may be a click-crosslinked network of ketone-and/or aldehyde-modified hyaluronan crosslinked with poly(ethylene glycol)-oxyamine. More particularly, the polymeric network may be a crosslinked polymeric network of poly(ethylene glycol)-tetra(oxyamine) and/or poly(ethylene glycol)-dioxyamine.

The proteins are any one or combination of IgG, IgA, IgD, IgE or IgM antibodies, and the antibody may be selected from the groupconsisting of, but not limited to, Adalimumab, Aducanumab, Alirocumab, Amivantamab, Anifrolumab, Ansuvimab, Avelumab, Belimumab, Cemiplimab, Daratumumab, Denosumab, Emapalumab, Enfortumab vedotin, Evinacumab, Lanadelumab, Necitumumab, Nimotuzumab, Nivolumab, Ofatumumab, Pamrevlumab, Pantitumumab, Ramicirumab, Secukinumab, Sotrovimab, Teprotumab, Alemtuzumab, Benralizumab, Bevacizumab, Bimekizumab, Crizanlizumab, Daclizumab, Dostarlimab, Emicizumab, Eptinezumab, Etaracizumab, Idarucizumab, Itolizumab, Natalizumab, Obituzumab, Ocrelizumab, Omalizumab, Pemrbolizumab, Pertuzumab, Tafasitamab, Tildrakizumab, Vedolizumab, Capromab, Fanolesomab, Ibritumomab tiuxetan, Imciromab, Moxetumomab pasudotox, Racotumomab, Cetuximab, Dinutuximab, Ertumaxomab, Isatuximab, loncastuximab tesirine, Oblitoxaximab, and Siltuximab.

The antibody may be bevacizumab, anti-sFRP2 or panitumumab.

The proteins may be fusion proteins comprising a portion of a Fc region of an IgG, IgA, IgD, IgE or IgM antibody, and the fusion protein may be Fc-fusion protein, such as Fc-noggin.

The peptidic ligand may comprise a chain extender, and the chain extender may be a poly(ethylene glycol), and as well the chain extender may be a non-binding amino acid sequence or it may be an amino acid sequence GAKSKG. The chain extender may be used to enhance peptide aqueous solubility and minimize steric hindrance effects.

The amino acid sequence that will bind to the Fc region of the antibody may comprise a sequence selected from the group consisting of HWRGWV, HYFKFD, HFRRHL, HWCitGWV, HWRGWVKGKASKG, (CFHH)₂KG, NVQYFAV, FYWHCLDE, FYTHCAKE, FYCHTIDE, GSYWYQVWF, RRGW and DCAWHLGELVWCT. In particular, the amino acid sequence may comprise HWRGWV, or it may comprise (CFHH)₂KG.

The proteins may be a mixture of two or more different proteins.

The peptidic ligands may be a mixture of two or more ligands having a different amino acid sequence. The proteins are present within the hydrogel and on the external surface of the hydrogel, and the hydrogel may be an injectable hydrogel.

A further understanding of the functional and advantageous aspects of the present disclosure can be realized by reference to the following detailed description and drawings.

### BRIEF DESCRIPTION OF THE DRAWINGS

Embodiments of the protein-release system for sustained release of proteins disclosed herein will now be described, by way of example only, with reference to the drawings, in which:

FIG. 1A shows the general structure of IgG antibodies.

FIG. 1B shows an affinity-based delivery system for controlling the release of antibodies. Fc binding ligands are in dynamic equilibrium with the Fc-domain of IgG, enabling sustained release of the antibody of interest from a hydrogel matrix.

FIGS. 2A and 2B are plots of wavelength shift versus time (s) showing affinity binding curves of Fc-Ligands, FcL1 and FcL2 to bevacizumab, respectively;

FIGS. 2C and 2D are plots of wavelength shift versus time (s) showing affinity binding curves of FcL1 and FcL2 to panitumumab, respectively;

FIGS. 2E and 2F are plots of wavelength shift versus time (s) showing affinity binding curves of FcL1 and FcL2 to anti-sFRP2, respectively and

FIGS. 2G and 2H are plots of wavelength shift versus time (s) showing affinity binding curves of FcL1 and FcL2 to Fc-noggin, respectively. Association and dissociation rates were determined by immobilizing biotinylated FcL-1 and FcL-2 onto high precision streptavidin biosensors (SAX) using the Octet red96 system.

FIG. 3 shows affinity interactions between FcL1 and human (h) IgG1 measured via bio-layer interferometry.

FIGS. 4A and 4B are schematics of the interaction between Fc-ligands and hIgG1. FIG. 4A shows the binding site of unmodified HWRGWV onto hIgG1. FIG. 4B shows the binding site of FcL1 onto hIgG1. Polar contacts between the binding pairs are shown as dotted lines with hlgG residues labeled.

FIGS. 5A and 5B show the synthesis of hyaluronan modified with ketone and tetrazine groups in which: FIG. 5A is a schematic of the synthetic pathway of HAKT: (1) 3-(2-methyl-1,3-dioxolan-2-yl)propan-1-amine is reacted with HA via amidation producing HA-ketal; (2) Methylphenyltetrazine amine is covalently bound onto HA-ketal through amidation; (3) The resultant polymer is deprotected in acidic conditions yielding HAKT, and FIG. 5B shows the 1H NMR measurements demonstrating the successful synthesis of HAKT.

FIGS. 6A and 6B show the characterization of the HAKT-oxime gel in which: FIG. 6A shows that HAKT-oxime gel is degradable in the presence of hyaluronidase at 10 IU/mL and 100 IU/mL, and FIG. 6B shows that HAKT-oxime gel is injectable by hand through a 30 G needle over 2 hours (n=4, mean±SD).

FIGS. 7A and FIGS. 7B show sustained release of anti-sFRP2 is achieved through the incorporation of FcL-1 into HAKT-oxime gels, in which:

FIG. 7A shows plots of cumulative release of anti-sFRP2 over a 7-day period from HAKT-gels embedding no FcL1 and FcL1 at 1:100 molar ratio, antibody:peptide (n=3, mean±SD plotted, two-way-ANOVA, Šidák post-hoc test, ****p<0.0001);

FIG. 7B Relative antisFRP2 diffusivity from HAKT-gel formulations determined using the short-time approximation for Fickian diffusion from a thin layer (mean±SD plotted, unpaired, two-tailed, Student's t-test, *p<0.05).

FIGS. 8A to 8D show sustained release of Fc-Noggin, in which:

FIG. 8A shows plots of cumulative release of Fc-Noggin from HAKT-gel containing no FcL1 and FcL1 (100×molar excess) (n=3, mean±SD plotted, two-way-ANOVA, Šidák post-hoc test, *p<0.05, **p<0.01);

FIG. 8B is a plot of the relative Fc-Noggin diffusivity through HAKT with and without FcL1 determined using the short-time approximation for Fickian diffusion from a thin layer (mean±SD plotted, unpaired, two-tailed, Student's t-test, *p<0.05);

FIG. 8C shows plots of cumulative release versus time (days) showing the 7-day release of Fc-Noggin from HAKT-gel into release media at pH 7.4, 9.0, and 10 (n=3, mean±SD plotted, two-way-ANOVA, Šidák post-hoc test, *p<0.05, **p<0.01, ****p<0.0001); and

FIG. 8D shows a plot of cumulative release of Fc-Noggin from HAKT-gel into PBS or PBS containing 250 mM NaCl (n=3, mean±SD plotted, two-way-ANOVA, Šidák post-hoc test, *p<0.05, **p<0.01, ****p<0.0001).

FIG. 9A shows plots of percent cumulative release versus time (days) showing the 7-day release of anti-sFRP2 from HAKT-oxime gel containing 1:100 molar ratio antibody:peptide and no peptide.

FIG. 9B shows plots of percent cumulative release versus time (s⁻¹) showing Fickian diffusion phases from release profiles in FIG. 9A. The slopes, m₁ and m₂, are proportional to apparent protein diffusivity through the gel (n=3, mean±SD, ****p<0.0005).

FIG. 10 shows the release of anti-sFRP2 from HAKT-gel containing no peptide and 100-fold molar excess FcL2.

FIG. 11 shows the number of primary RSC spheres arising from CE-derived cells cultured at a clonal density of 10 cells per microliter. Cells are grown in serum free media (SFM); in the presence of sFRP2 (untreated); and in the presence of sFRP2 with fresh antisFRP2, antisFRP2 incubated at 37 C for 7 days, released antisFRP2 from HAKT gel without FcL1, and released antisFRP2 from HAKT gel embedding 100-fold molar excess FcL1 to antibody (mean±SD plotted, one-way-ANOVA, Šidák post-hoc test, **p<0.01).

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms, “comprises” and “comprising” are to be construed as being inclusive and open ended, and not exclusive. Specifically, when used in the specification and claims, the terms, “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. In one non-limiting example, the terms “about” and “approximately” mean plus or minus 10 percent or less.

Unless defined otherwise, all technical and scientific terms used herein are intended to have the same meaning as commonly understood to one of ordinary skill in the art.

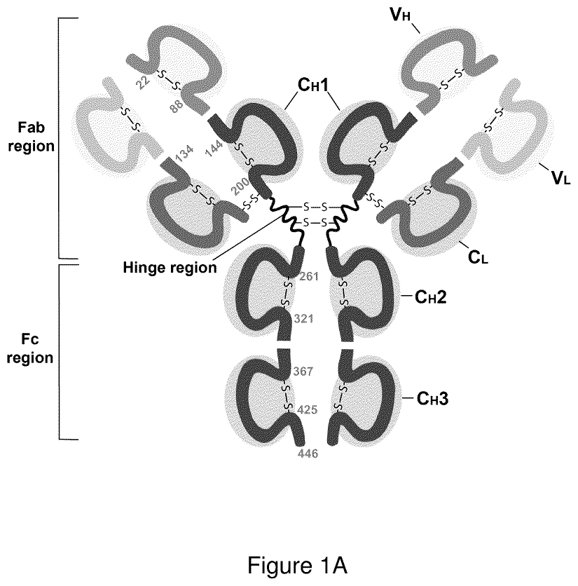

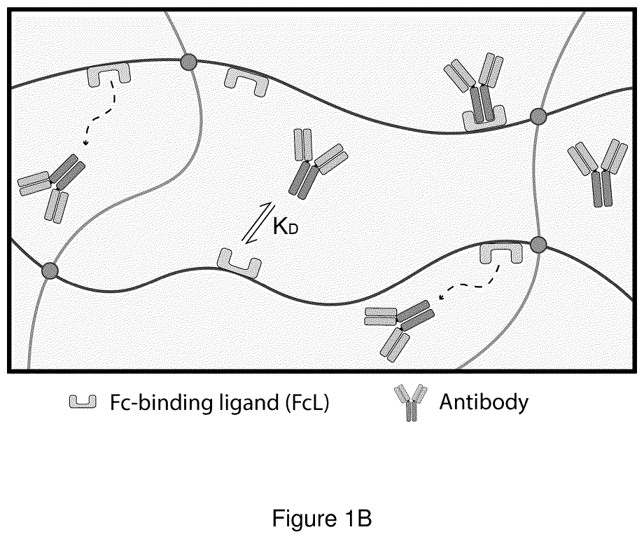

Referring to FIG. 1A, the IgG immunoglobulin molecule is comprised of four polypeptide chains containing identical pairs of light chains and heavy chains forming a flexible Y-shaped structure linked by inter-chain disulfide bonds. Each heavy chain contains a variable region (VH) at the N-terminus and three constant regions (CH1, CH2, CH3) with a hinge region connecting CH1 and CH2. The light chains comprise a variable domain (VL) located at the N-terminus, and a constant domain (CL). The association of the light chain with the VH and CH1 regions form the fragment antigen binding domain (Fab). The lower hinge region is comprised by the CH2 and CH3 domains forming the fragment crystallizable domain (Fc). The main difference in the functional characteristics of different antibody isotypes lie mainly in the Fc fragment and hinge regions, hence determining their functional properties. The numbers along the light and heavy chains represent the amino acid positions.

FIG. 1B shows a diagrammatic representation of a physiologically acceptable hydrogel showing peptidic ligands bound to the polymeric network in various locations which the antibodies of interest are reversibly bound to by affinity of the Fc region to the peptidic ligands.

The protein-release system and method disclosed herein will now be illustrated using the following non-limiting and exemplary examples.

According to an embodiment, the protein-release system may comprise a mixture of two or more different proteins, each protein having a selected binding affinity with the peptidic ligands. Alternatively, the protein-release system may comprise a mixture of two or more different proteins and the peptidic ligands are a mixture of two or more different peptidic ligands, each protein having a selected binding affinity with each peptidic ligands. Alternatively, the protein-release system may comprise a mixture of two or more ligands having a different amino acid sequence and binding affinity such as the protein binds with different affinities to the different ligands. Such approaches may allow a user to modify, adjust and/or tailor the release.

According to an embodiment, the proteins are not only present within the hydrogel but also on the external surface of the hydrogel.

### Materials and Methods

### Materials

All reagents were used as received unless otherwise stated. The following chemicals were purchased from Sigma-Aldrich: Dichloromethane (DCM), dimethylformamide (DMF), n-methyl-2-pyrrolidone (NMP), acetonitrile (ACN), ethyl ether, tetrakis(triphenylphosphine)palladium(0), borane-dimethylamine complex, 5-norbornene-2-carboxylic acid (mixture of isomers), piperidine, triisopropylsilane (TIS), trifluoroacetic acid (TFA), ethanedithiol, sodium phosphate monobasic monohydrate, sodium phosphate dibasic heptahydrate, Fmoc-Cys(trt)-OH, Fmoc-Lys(Alloc)-OH, Fmoc-Lys(Boc)-OH, Fmoc-Gly-OH, Fmoc-His(trt)-OH, Fmoc-Gly-Wang resin (100-200 mesh), and bovine serum albumin (BSA). (Boc-aminooxy)acetic acid, 4-(4,6-Dimethoxy-1,3,5-triazin-2-yl)-4- methylmorpholinium chloride (DMT-MM) were obtained from TCI chemicals. N,N′-diisopropylcarbodiimide and 5-norbornene-2-methylamine (a mixture of isomers) were purchased from Tokyo Chemical Industry.

The following materials were ordered from R&D systems: Mouse sFRP-2 Antibody (anti-sFRP2), Human VEGF (Research Grade Bevacizumab Biosimilar) Antibody, Recombinant Mouse Noggin Fc Chimera Protein (Fc-noggin) and Recombinant Mouse Sfrp2 Protein. The following materials were obtained from ThermoFisher Scientific: HRP-conjugated Goat anti Mouse IgG Fc Secondary, Pierce™ SuperBlock™ Blocking Buffer, Pierce™ Quantitative Fluorometric Peptide Assay. The following chemicals were obtained from Anaspec Inc.: Fmoc-Ala-OH, Fmoc-Arg(Pbf)-OH, Fmoc-Ser(tBu)-OH, Fmoc-Trp(Boc)-OH, and 1-Hydroxybenzotriazole (HoBt). Recombinant Human VEGF 165 Protein (Novus Biologicals), Panitumumab (Abmole Biosciences), Sodium hyaluronate (309 kDa) (Lifecore Biomedical), poly(ethylene glycol)-tetra(amine) average Mn 5250 Da (Jenkem Technology), Fmoc-lys(biotin)-OH (ChemPep), methyltetrazine-amine HCl salt (Click Chemistry Tools), EDTA-free protease inhibitor tablets (Roche). Methyl cellulose dialysis membranes of 1 kDa and 12-14 kDa cut-off were purchased from Spectrum Laboratories.

### Biotin-Modified FcL-1 and FcL-2

HWRWVGAK′SKG (FcL-1-Biotin) and (CFHH)₂KGGAK′SKG (FcL-2-Biotin), K′=Fmoc-lys(biotin)-OH, were prepared via the Liberty Blue™ Automated Microwave Peptide Synthesizer using standard solid phase synthesis on Fmoc-Gly-Wang Resin. The terminal Fmoc was deprotected in a DMF mixture comprising 20% piperidine and 0.1 M HoBt for 1 h at room temperature. The reaction solution was decantated, and the resin was washed with DMF and DCM and allowed to dry. Biotin-modified peptides were cleaved off the resin using a cocktail containing either; 95% TFA, 2.5% water and 2.5% triisopropylsilane (TIS) for FcL-1-Biotin; or 94% TFA, 2.5% water, 2.5% ethanedithiol, and 1% TIS for FcL-2-Biotin. After resin cleavage, peptides were purified through a C18 column (1:3 ACN:H2O). Fractions were collected and analyzed via ESI/MS. Biotin-modified peptide fractions were allowed to dry for 48 h and lyophilized for 3 days.

### Biolayer Interferometry

Binding kinetic measurements were performed on the Octet RED96 system (ForteBio, CA) using single-use high-precision streptavidin biosensors (SAX). Assay buffer was defined as 1×PBS comprising 0.05% Tween-20 (PBST). Biotinylated peptides FcL-1 and FcL-2 were immobilized at 100 nM. Non-specific interactions were minimized by including a blocking step using SuperBlock prior to each baseline response. Bevacizumab, panitumumab, anti-sFRP2 and Fc-Noggin were diluted into assay buffer at the specified concentrations typically 350-0 nM and allowed to associate for 600 s followed by dissociation for 600 s. A double-referencing experiment was used to assess the binding interactions of Fc-noggin to both peptides. Peptides were immobilized in sample sensors while reference sensors were dipped in assay buffer. For association, the sample and reference sensors were dipped into Fc-noggin. All experiments included one biosensor dipped in assay buffer during association for background normalization. Data was analyzed and fitted by applying the 1:1 binding model for bevacizumab and panitumumab and the 2:1 binding model for anti-sFRP2 and Fc-noggin as implemented in the ForteBio Data Analysis suite.

### Norbornene-Modified FcL-1 and FcL-2

Modified Fc-binding peptides, HWRWVGAKSK″G (FcL-1) and (CFHH)₂KGGAKSK″G (FcL-2), K″=Fmoc-lys(alloc)-OH, were prepared via the Liberty Blue™ Automated Microwave Peptide Synthesizer using standard solid phase synthesis on Fmoc-Gly-Wang Resin. An alloc-protected lysine was added in the C-terminus to provide an amine for norbornene functionalization. First, Tetrakis(triphenylphosphine)palladium(0) (57.9 mg, 0.05 mmol) and borane dimethylamine (59.0 mg, 1 mmol) were added to the mixture using DCM (8 mL) and allowed to react for 1 h. The solution was discarded, and the resin was washed with DCM several times to remove excess reagents. Norbornene-2-carboxylic acid (207.2 mg, 1.5 mmol) was activated with N,N′-Diisopropylcarbodiimide (378.6 mg, 3 mmol) in a 1:1 DCM:n-methyl-2-pyrrolidone (10 mL) mixture for 20 minutes at room temperature. Subsequently, the activated solution was added to the resin-bound peptide and allowed to react overnight. The resin was washed with DCM as described above. Then, the N-terminal Fmoc was deprotected using a solution containing 20% piperidine and 0.1 M HoBt in DMF (10 mL) for 1 h at room temperature. The reaction solution was decantated, and the resin was washed with first with DMF and then DCM. The resin was allowed to dry overnight under air-flow.

The norbornene-modified peptide was cleaved off the resin using a cocktail containing either; 95% TFA, 2.5% water and 2.5% triisopropylsilane (TIS) for FcL-1; or 94% TFA, 2.5% water, 2.5% ethanedithiol, and 1% TIS for FcL-2. After resin cleavage, the supernatant was decanted into a falcon tube containing cold ethyl ether (0° C., 40 mL) and centrifuged for 3 minutes at 2000 rpm. The supernatant was discarded and the sedimented product was washed one more time with cold ethyl ether and centrifuged. The supernatant was removed, and the product was allowed to dry overnight. Peptides were purified through a C18 column (1:3 ACN:H₂O). Fractions were collected and analyzed via ESI/MS. Norbornene-modified peptide fractions were allowed to dry for 48 h and lyophilized for 3 days.

### Ketone-Substituted Hyaluronan (HAKetal)

HAKetal was prepared as previously described. In brief, sodium hyaluronate (1.00 g, 309 kDa) was dissolved in 2-(N-morpholino)ethanesulfonic acid (MES) buffer (100 mL, 0.1 M in water, pH 6.6) and mixed with DMTMM (1.72 g, 6.2 mmol). After 20 min, 3-(2-methyl-1,3-dioxolan-2-yl)propan-1-amine (0.36 g, 2.49 mmol) was added dropwise and allowed to react for 48 h at room temperature. Then, the solution was dialyzed against 0.1 M NaCl using methyl cellulose dialysis membrane (12-14 kDa MWCO) for 48 h followed by distilled water for 24 h. The resultant polymer was lyophilized and analyzed by ¹H NMR in deuterium oxide to determine ketal substitution (38%).

### Tetrazine-Modified Hyaluronan Ketone (HAKT)

HAKetal (1.00 g) was dissolved in MES buffer (100 mL, 0.1 M in water, pH 6.6) and stirred with DMTMM (30.8 mg, 1.12 mmol). After 20 minutes, methyltetrazine-amine HCl salt (44.96 mg, 0.22 mmol) was added and allowed to react for 3 days at room temperature. The mixture was dialyzed against 0.1 M NaCl for 24 h followed by 2 h in 0.1 M HCl. The solution was changed to pH 4.5 with a 0.025 M sodium phosphate solution for 24 h. The purified polymer was sterile filtered (0.22 um, Millipore) and lyophilized. HAKT was characterized by ¹H NMR in deuterium oxide to determine tetrazine substitution (7.7%).

### PEG-Tetra(Oxyamine) (PEGOA4)

PEGOA4 was synthesized as previously reported. Briefly, (Boc-aminooxy)acetic acid (0.32 g, 1.67 mmol) was dissolved in 25 mL of anhydrous DCM and mixed with N,N′-diisopropylcarbodiimide (DIC) (0.44 mL, 2.8 mmol) at 0° C. under nitrogen for 1 h. Then, PEG-tetramine (1.00 g, 5250 Da) was added to the mixture followed by N,N-diisopro-pylethylamine (0.54 mL, 3.1 mmol) and allowed to react at room temperature for 48 h. Subsequently, the solvent was removed in through a rotary evaporator and the crude was mixed with distilled water and filtered through a 0.22 μm filter to remove the N,N′-diisopropylurea byproduct. The resultant filtrate was dialyzed against 0.1 M NaCl for 24 h followed by distilled water for 48 h using methyl cellulose dialysis membranes (1 kDa MWCO). The dialysate was lyophilized for 3 days. The substitution of Boc-protected PEG-tetra(oxyamine) was characterized by ¹H NMR. The polymer was dialyzed against 0.2 M HCl for 48 h, to remove the Boc-protecting group, followed by distilled water for 48 h. The dialysate was lyophilized for 3 days to yield PEG-tetra(oxyamine) (PEGOA₄). A sample was analyzed by ¹H NMR in deuterated chloroform to confirm deprotection.

### Preparation of HAKT-Gels

HAKT-hydrogel was synthesized as follows: HAKT (20 mg) and PEGOA4 (100 mg) were dissolved separately in PBS (1 mL). Once in solution, both polymers were mixed to yield a final formulation comprising 1.25 wt % HAKT and 1.19 wt % PEGOA4 (80 mol % crosslinking). Pre-gels were speedmixed for 30 seconds maximum speed and incubated at 37° C. for gelation to occur.

FcL-1 immobilization onto HAKT

To prepare HAKT-FcL-1, HAKT was dissolved in PBS at 4 mg/mL, mixed with norbornene-modified FcL-1 at a final concentration of 0.2 mg/mL and allowed to react overnight at room temperature. The modified polymer was then dialyzed in 0.1 M NaCl using methyl cellulose dialysis membrane (12-14 kDa molecular weight cut-off) for 24 h followed by 48 h in distilled water. The dialysate was sterile filtered through a 0.22 um syringe filter and lyophilized for 3 days. The degree of substitution was determined using Pierce™ Quantitative Fluorometric Peptide Assay (Thermo Scientific).

### Rheological Characterization

HAKT was dissolved at 20 mg/mL in PBS. PEGOA4 was dissolved at 109.5 mg/mL in PBS. HAKT was mixed with PEGOA4 to yield a final concentration of 12.5 mg/mL and 11.9 mg/mL, respectively. Time sweep measurements of HAKT-hydrogels were assessed using an AR-1000 rheometer fitted with a 20 mm, flat plate (TA instruments) at 2% strain and 1 Hz for 2 h using a solvent trap to reduce evaporation. HAKT-oxime mixtures of 100 μL were loaded on the rheometer at 37° C. Time sweep plots were used to calculate the gelation time, when the storage modulus (G′) crossed the shear loss modulus (G″). Experiments were performed in triplicates.

### In Vitro Stability

100 uL aliquots of HAKT-oxime hydrogels were dispensed in pre-weighed 2 mL Eppendorf tubes overnight at 37° C. Hydrogel mass was taken after swelling the hydrogels with 400 μL of PBS for 2 h, 6 h, 1 d, 2 d, 3 d, 7 d, 14 d, 21 d and 28 d. At each time point the supernatant was removed, the hydrogel mass was recorded and then replaced with fresh media. The mass ratio was determined as the ratio of hydrogel mass at the time point to the initial mass.

### Injectability

The injectability of HAKT-oxime gels was quantitatively assessed by using the Series 5 advanced digital force gauge (Mark-10). HAKT-oxime gels containing 1.25 wt % HAKT and 80 mol % PEGOA4 were dispensed in a 1 mL syringe prior to injection. The loading force required to displace the plunger was measured using a 23 G, 27 G and 30 G needle at different time points (10 min, 20 min, 40 min, 1 h, 1.5 h and 2 h). Measurements were performed in triplicate.

### Controlled Release of Bevacizumab and Anti-sFRP2

HAKT and HAKT-FcL-1 were dissolved in PBS to yield a final HA content of 12.5 mg/mL with 0 or 100 molar excesses of peptide to protein and mixed with PEGOA4 at 80 mol %. Bevacizumab (4 ug), anti-sFRP2 (1 ug), or Fc-Noggin (1 ug) were incorporated into 100 μL of gel and incubated at 37° C. for 2 h to allow gelation. Release media was defined as 1×PBS comprising 0.1 wt % bovine serum albumin and Roche cOmplete™ Protease Inhibitor Cocktail (1 tablet/ 10 mL, Roche, Switzerland). Protein release was evaluated by incubating HAKT-oxime gels with 400 μL of release media on an orbital shaker at 37° C. Supernatants were collected and replaced with fresh release media after 2 h, 6 h, 1 d, 2 d, 3 d, 5 d, and 7 d. HAKT-oxime hydrogels were digested after the last time point using 500 IU/mL hyaluronidase in 400 μL of PBS. Fc-Noggin concentrations were evaluated using a mouse Noggin ELISA (RayBiotech). A custom-made ELISA was developed and validated to determine the amount released of anti-sFRP2 and bevacizumab.

### ELISA

Hisorb Ni-NTA 96-well plate (Qiagen, Toronto, ON) wells were coated with 200 μL of a 0.5 mg/mL VEGF solution or a 1.2 ug/mL recombinant mouse sFRP2 solution in PBS at 4° C. overnight. Subsequently, the wells were washed with 200 μL TPBS (1× PBS with 0.05% Tween 20) four times and blotted dry on paper towels. Standard and analyte samples were added to each well (100 μL) and incubated for 2.5 h at room temperature on an orbital shaker. The wells were then washed as previously described and blotted dry. Anti-Fc antibody was dissolved in PBSA (0.1% BSA in 1× PBS) at 1:100,000 or 1:8,000 for bevacizumab or anti-sFRP2 detection, respectively. 100 μL of anti-Fc antibody diluted at appropriate concentrations was added to each well and incubated for 1.5 h at room temperature. A solution of 3,3′,5,5′-Tetramethylbenzidine (TMB) was added (100 ul) to each well. The plate was covered in aluminum foil and incubated at room temperature on an orbital shaker (bevacizumab—10 min, anti-sFRP2—30 min). Then, a stop solution comprising 2N H₂SO₄ (50 μL) was added to each well and the absorbance was measured at 450 nm. Protein concentration was determined using the linear range in the standard curve. Protein samples were diluted to fit within the linear range.

Calculation of Protein Diffusivity from Hydrogels

A unidirectional diffusion from a plane sheet was used to compare the release rates from HAKT-oxime and HAKT-oxime-FcL-1 using the equation:

$$\frac{{M}_{t}}{{M}_{∞}} = {mt}^{1 / 2}$$

Where Mt is the cumulative mass of drug released at time t, M_{∞} is the cumulative mass of drug released as time approaches infinity, and m is the constant proportional to apparent diffusivity of protein within the gel. The slope of the linear segment k, representing Fickian Diffusion, was determined by plotting the ratio of Mₜ to M_{∞}. Protein diffusivity was then determined using the following equation:

$$k = 4 {( \frac{D}{π {l}^{2}} )}^{1 / 2}$$

Where D is the protein diffusivity from the gel and l is the thickness of the gel.

### Mass Spectrometry

Mass spectrometry experiments were conducted at the Advanced Instrumentation for Molecular Structure (AIMS) core facility at the University of Toronto. Purified peptides were dissolved in PBS and ionized using ESI.

### Statistical Analysis

All data are presented as a mean±standard deviation. Statistical analyses were performed using GraphPad 9.1.3 (GraphPad Software, San Diego, CA). Differences between groups were assessed using two-way ANOVA and Student's t-test as appropriate.

Results and discussion
Affinity interactions

We investigated two peptides previously shown to bind specifically to the fragment crystallizable domain (Fc): HWRGWV (Yang et al., 2010) and (CFHH)₂KG (Verdoliva et al., 2005). These peptidic ligands were modified with a spacer group, GAKSKG, in the C-terminus to minimize steric hindrance effects that might affect interactions between the binding pair. Additionally, this spacer contains a lysine residue in proximity to the C-terminus to provide an amine for further chemical modification, enabling the insertion of different functional groups.

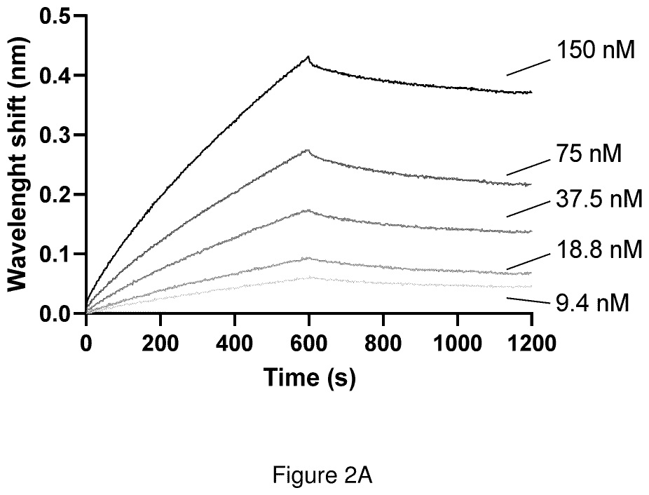

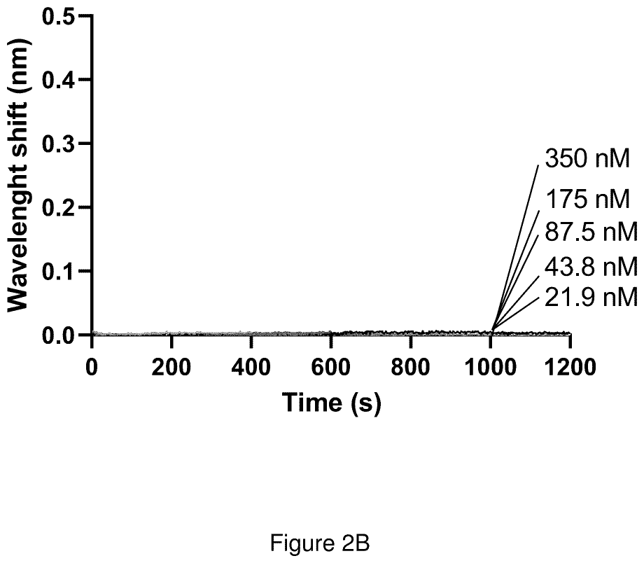

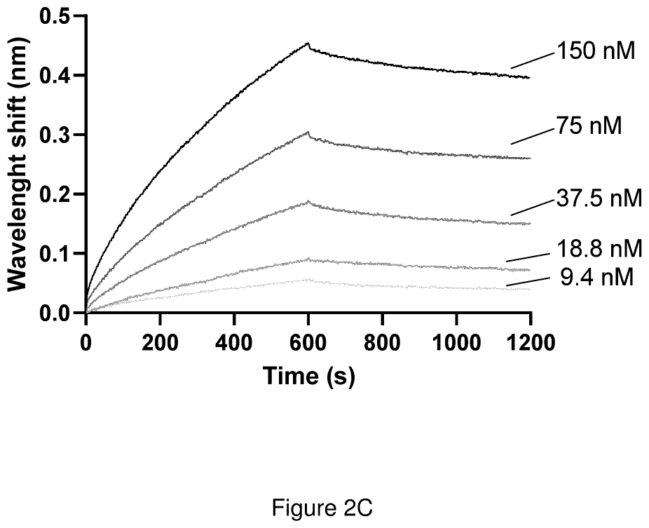

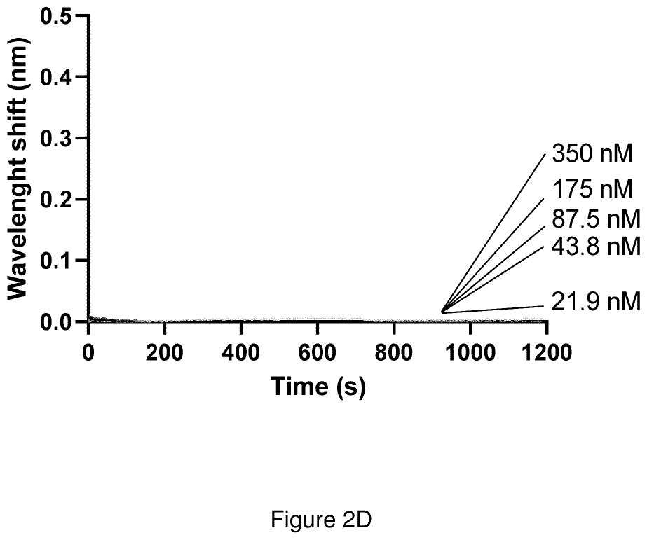

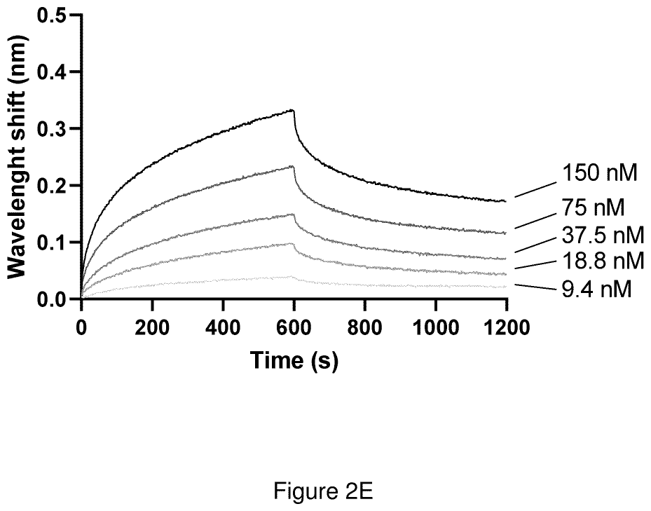

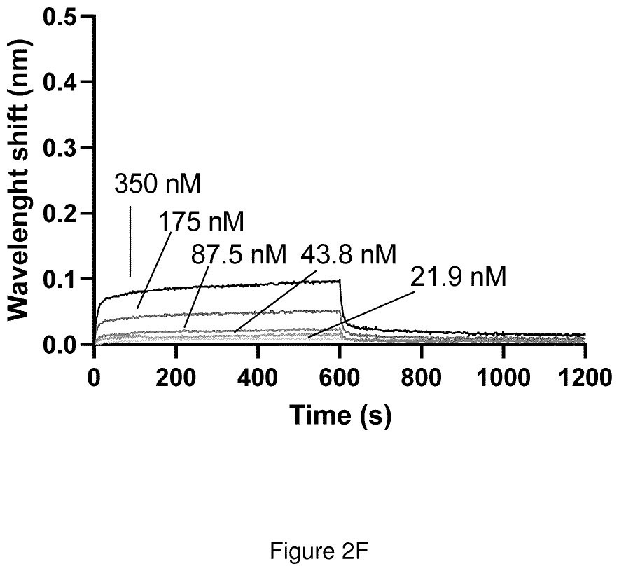

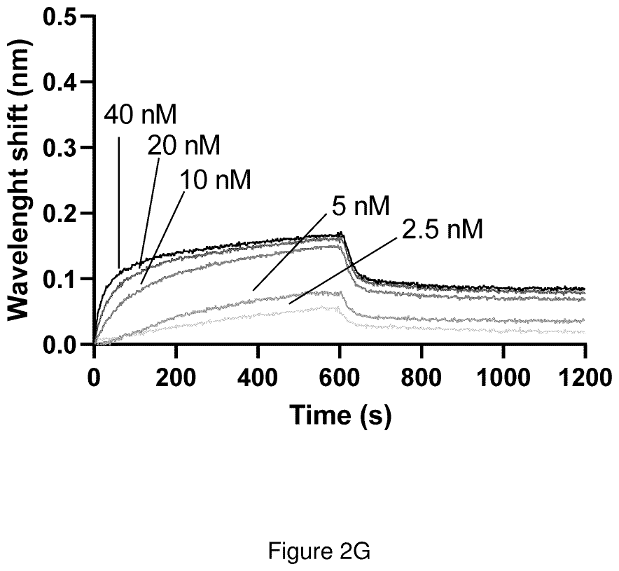

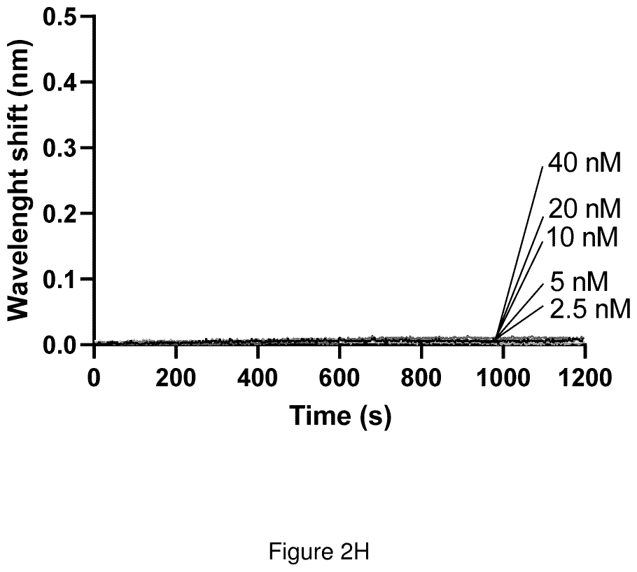

Affinity interactions of the modified peptide ligands to different antibodies were evaluated via bio-layer interferometry. Biotin-modified HWRGWVGAKSKG (FcL-1) and (CFHH)₂KGAKSKG (FcL-2) were immobilized onto high precision streptavidin biosensors (SAX) at 100 nM and subjected to different concentrations of bevacizumab, panitumumab, anti-sFRP2, or Fc-Noggin. FIGS. 2A to 2H show the binding profiles of FcL1 or FcL2 to bevacizumab (FIG. 2A-2B), panitumumab (FIG. 2C-2D), anti-sFRP2 (FIG. 2E-2F), and Fc-Noggin (FIG. 2G-2H). Dissociation constants between the binding pairs were determined using the Octet Data Analysis Software. Binding assays for bevacizumab and panitumumab were best represented by a 1:1 model, using analyte binding to one ligand site. A heterogeneous 2:1 ligand binding model was used for the evaluation of the equilibrium dissociation constants of the chosen peptide ligands to anti-sFRP2 or Fc-Noggin. This 2:1 binding model assumes analyte binding at two independent ligand sites, to which the analyte possesses distinct rate constants. The biphasic nature of the curves demonstrates this behavior: an initial fast on-rate followed by a slower on-rate is evidenced in the association step, while an initial fast dissociation followed by a slower off-rate is noted in the dissociation step.

All assays were optimized to minimize any possible non-specific interactions by including a blocking step using SuperBlock™. High non-specific binding can result in non-ideal binding profile in a kinetic assay, displaying an apparent heterogenicity. Interestingly, while FcL1 was shown to bind all four antibodies tested—bevacizumab, panitumumab, anti-sFRP2, and fusion protein, Fc-Noggin, FcL2 demonstrated binding only to anti-sFRP2. The affinity of bevacizumab, panitumumab, and Fc-Noggin for FcL2 may be hindered due to the nature of the antibody products. Together, these findings suggest that FcL-1 could be leveraged for the controlled delivery of various antibodies via non-covalent interactions with their Fc domain.

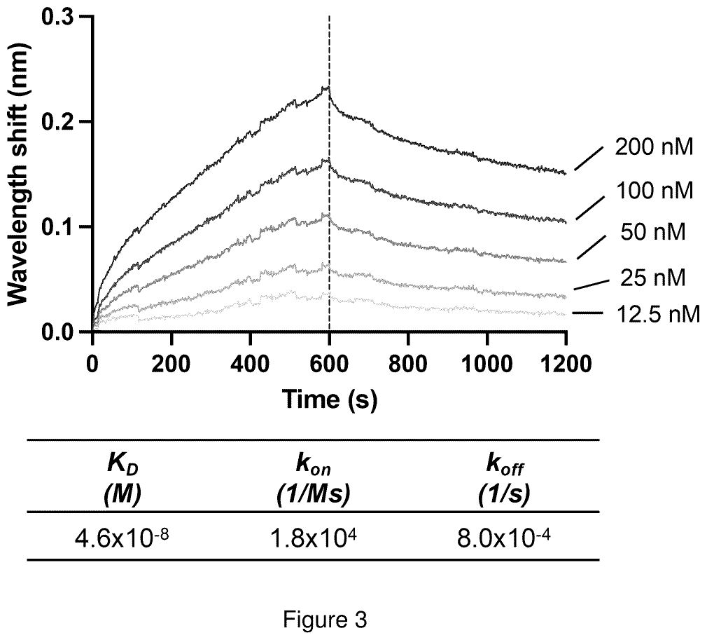

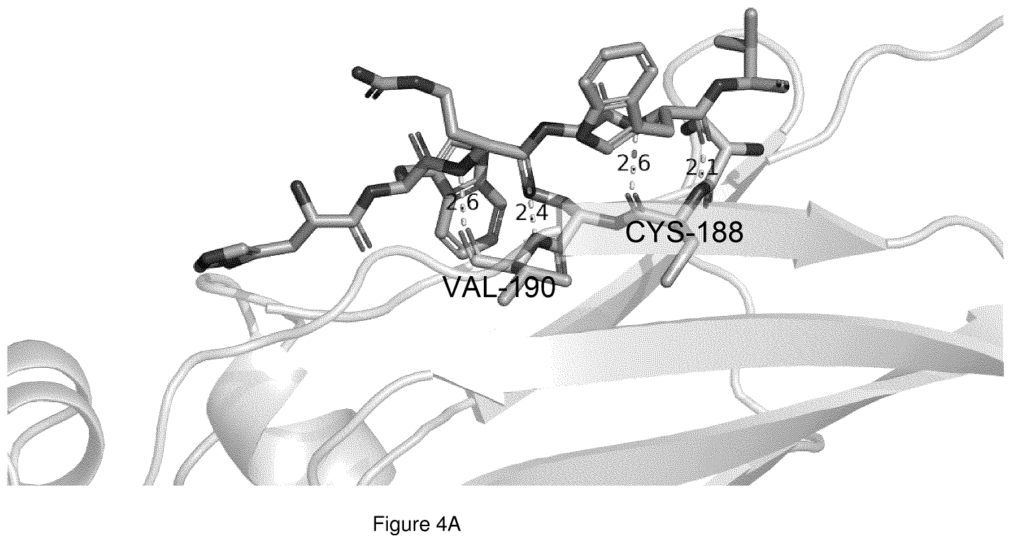

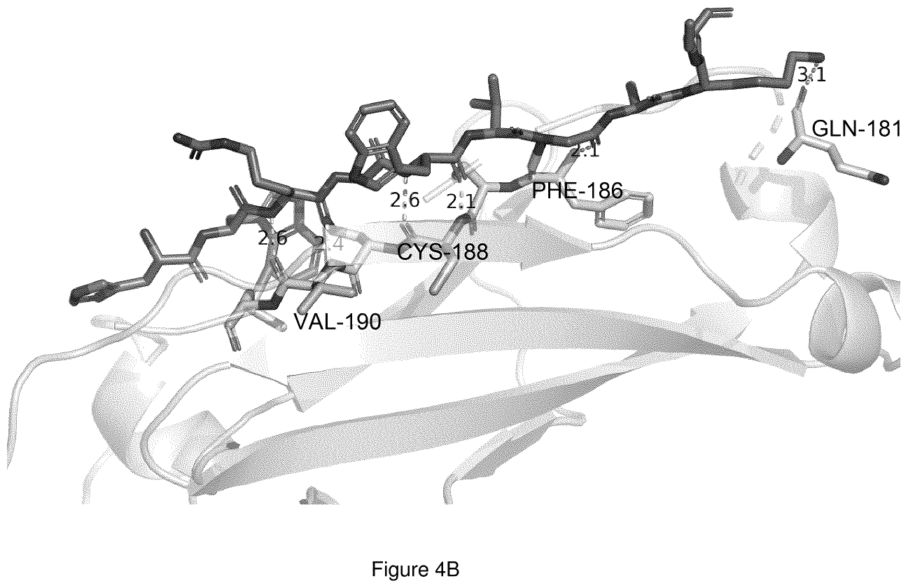

The unmodified peptide sequence, HWRWGV, was previously shown to possess a K_{D} value of 1.42×10⁻⁵ M when interacting with hlgG₁ (Reese et al., 2020). Binding kinetic results suggest that addition of the GAKSKG spacer to this peptide sequence led to a higher affinity interaction (K_{D}=4.6×10⁻⁵ M) (FIG. 3). To understand this behavior, we modeled the interaction between the Fc domain of hIgG1 to both HWRGWV and FcL1. In the predicted binding models, amino acids His1, Arg3, and Trp5 form polar interactions with Cys188-Val 190, located in the pFc′ region of hlgG₁ (FIG. 4A). This prediction is consistent with enzyme digests of hlgG found to bind HWRGWV columns (Yang et al., 2010). We then modeled the FcL1/Fc complex to find amino acid residues involved in the interaction. It was found that in addition to His1, Arg3, and Trp5, amino acids Glu7 and Lys9 are in close proximity to Gln181-Phe186 (FIG. 4B). These results suggest that the interaction of Glu7 and Lys9, in the GAKSKG spacer, with the Fc domain may increase the binding affinity of the Fc ligand.

### Physicochemical Properties of HAKT-Oxime Gel

A hydrogel system was utilized as a way to control release by incorporating selected Fc peptide ligands within the polymeric backbone. To facilitate independent control of the mechanical and biomolecular parameters, we engineered an injectable hydrogel system that takes advantage of two biorthogonal chemistries: oxime reaction and inverse electron demand Diels-Alder (IEDDA) ligation. Both, oxime and IEDDA reactions require no catalysts, and can be carried out under physiological conditions, thereby rendering this system amenable for protein delivery. In particular, we designed an oxime crosslinked hydrogel comprising poly(ethylene glycol)-tetraoxyamine (PEGOA4) and hyaluronan-ketone-tetrazine (HAKT) to which norbornene-modified FcL-1 was covalently bound.

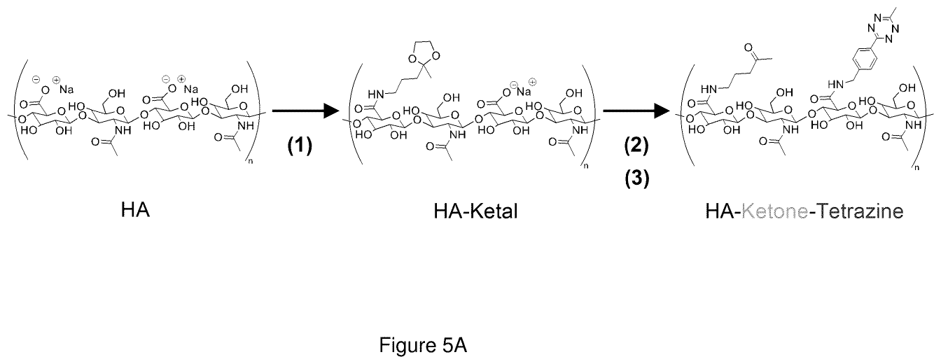

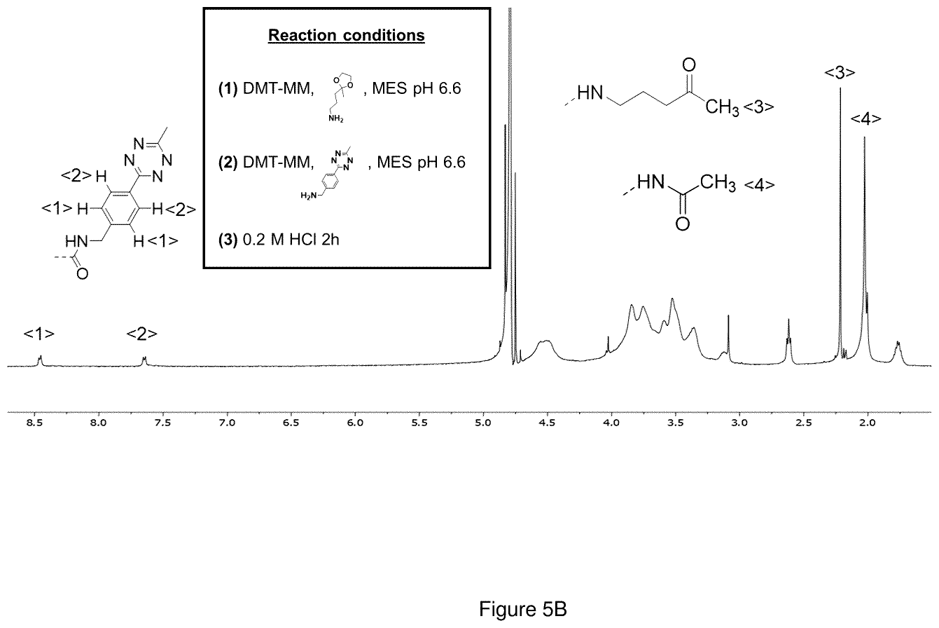

HAKT was synthesized using a three-step process (FIG. 5A). First, carboxylate groups in the polymer backbone were activated with 4-(4,6-dimethoxy-1,3,5-triazin-2-yl)-4-methylmorpholinium chloride (DMTMM). Subsequently, amide coupling was performed by conjugating 3-(2-methyl-1,3-dioxolan-2-yl)-1-propanamine with the activated polymer, resulting in HA-ketal. To functionalize HA-ketal with tetrazine, the remaining carboxylate groups were reacted with methylphenyltetrazine-amine using DMTMM as an activator. Finally, the ketal group was deprotected using mild acidic conditions, resulting in HAKT. The ketone and tetrazine substitution in HAKT were confirmed by ¹H NMR and determined to be 5.5% and 39%, respectively (FIG. 5B).

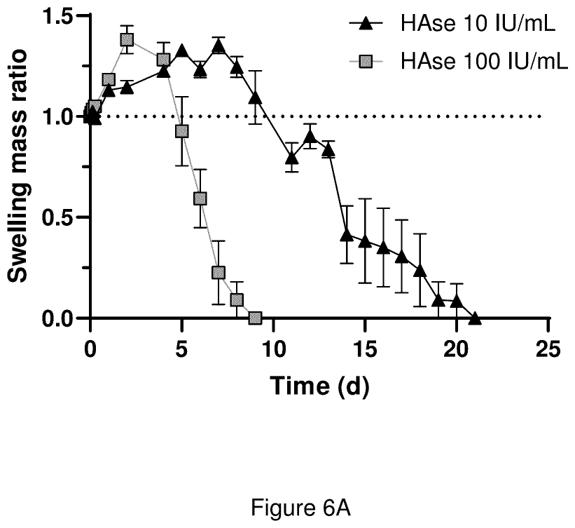

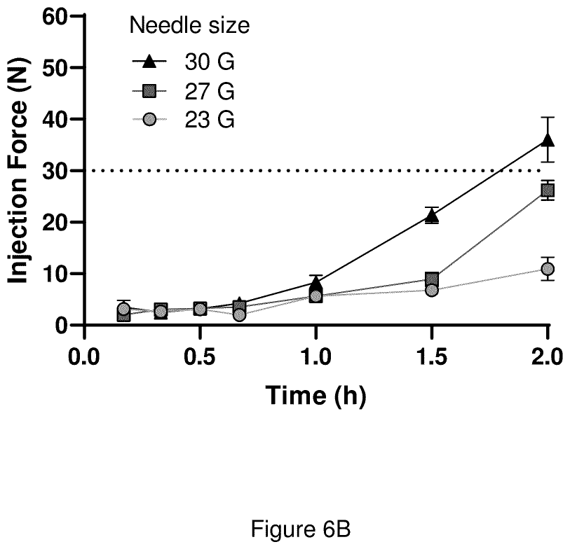

The inventors formulated HAKT-oxime gels to be comprised of 1.25 wt % HAKT and 1.19 wt % PEGOA₄ (80 mol % crosslinking). Gelation time was determined using a rheometer and determined to be approximately 35 minutes. This delivery vehicle was designed specifically to contain HA, enabling natural degradation with hyaluronidase. The inventors studied the in vitro degradability of HAKT-oxime gels using hyaluronidase (HAse) at 100 IU/mL and 10 IU/mL (FIG. 6A), results were normalized to control gels incubated with PBS only. Gels were degraded after 9 days and 20 days in the presence of HAse at 100 IU/mL and 10 IU/mL, respectively. Changes in hydrogel mass can be attributed to the enzymatic degradation of the HA backbone as well as the hydrolysis of the oxime linkage (Baker et al., 2021).The injectability of HAKT-oxime gels was assessed using a 1 mL syringe coupled to different needle sizes, 30 G, 27 G, and 23 G (FIG. 6B). The selected formulation is readily injectable by hand for up to two hours through a 30 G needle after which it surpasses the maximum threshold of injectability, 30 N, measured via the Series 5 advanced digital force gauge. Although HAKT-gels have a gelation time of 35 minutes, maximum storage modulus is only achieved after 4 hours, indicating that oxime bonds are still forming at 2 hours.

### Affinity-Controlled Release

As a proof-of-concept we investigated the affinity release of anti-sFRP2, Fc-Noggin and bevacizumab from a hydrogel matrix modified with FcL-1. We hypothesized that transient interactions between the binding pair would enable the controlled release of both antibodies. Norbornene-conjugated FcL-1 was covalently bound to HAKT via IEDDA conjugation. Substitution was evaluated via amino acid analysis as well as Pierce™ Quantitative Fluorometric Peptide Assay and determined to be 2.93 nmol/mg HAKT and 2.83±1.33 peptide/mg HAKT., respectively.

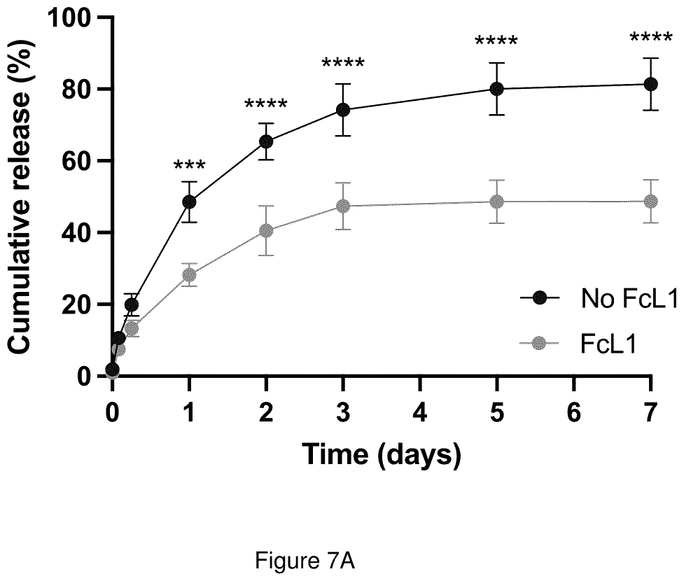

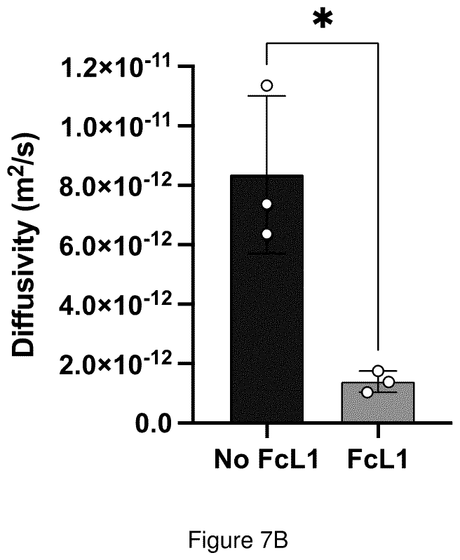

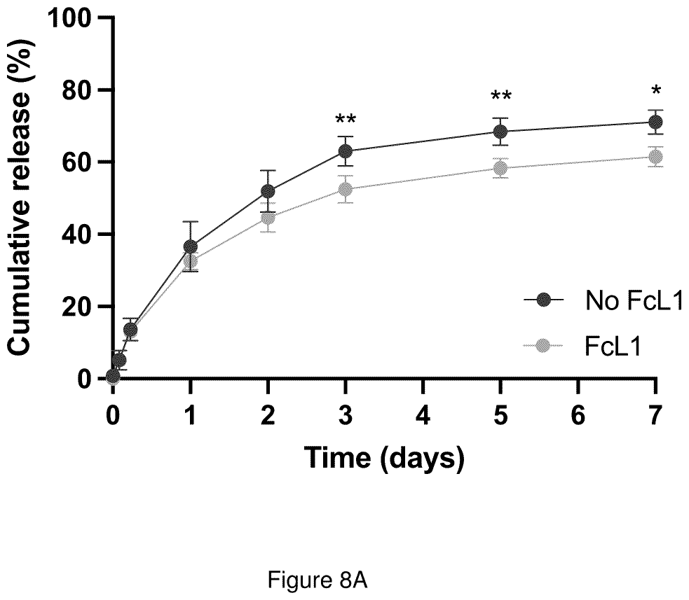

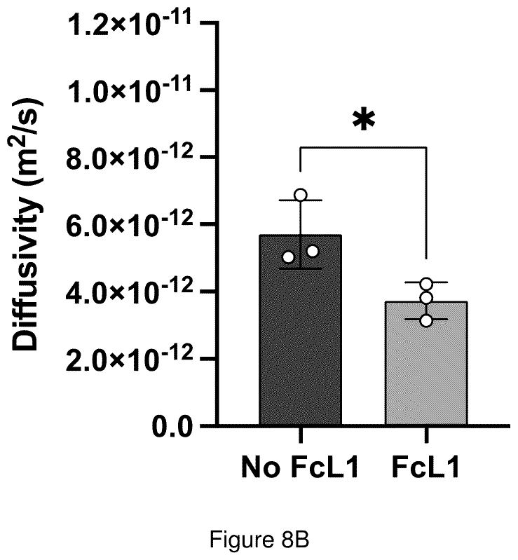

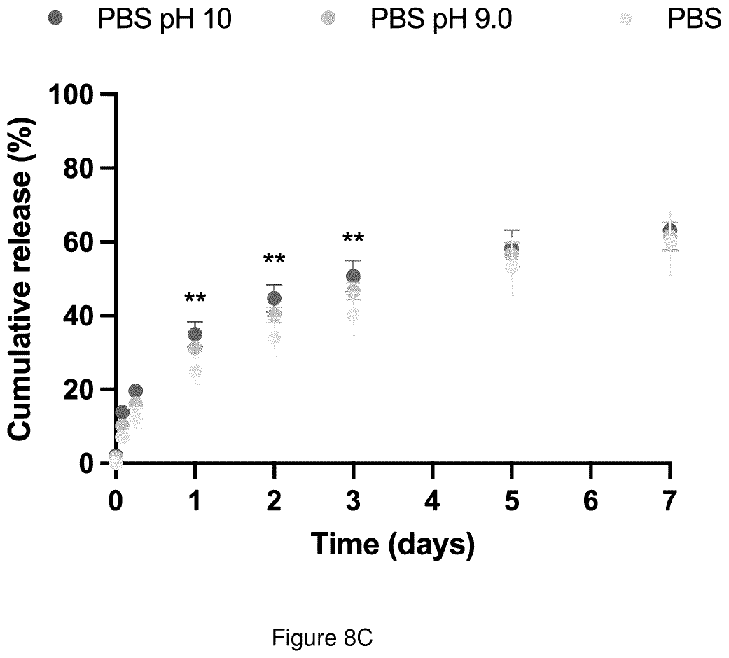

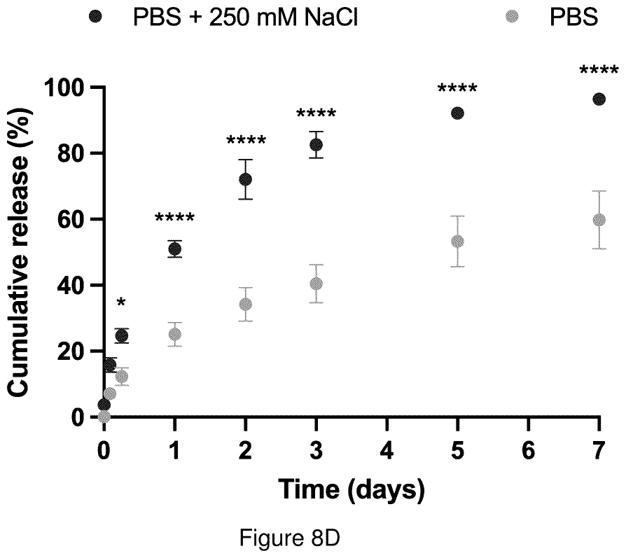

To test the effect of FcL-1 on antibody release, we prepared HAKT gels containing no peptide or 100-fold molar excess peptide and assessed the release profile of anti-sFRP2, Fc-Noggin and bevacizumab in vitro. Formulations were incubated at 37° C. in release media. At appropriate intervals, release media was completely removed and replaced with fresh media. Antibody release was quantified using ELISA. As shown in FIG. 7A, controlled release of anti-sFRP2 was achieved in the presence of FcL-1. Effective anti-sFRP2 diffusivity through the hydrogel was calculated using a short-time approximation for unidirectional diffusion from a thin slab (FIG. 7B). Results suggest that effective anti-sFRP2 diffusivity from HAKT-gel (8.4×10⁻¹²±2.6×10⁻¹² m²/s) was minimized with the incorporation of FcL-1 (1.4×10⁻¹²±3.6×10⁻¹³ m²/s). Similarly, controlled release of Fc-Noggin was achieved via incorporation of FcL-1 onto HAKT-gel (FIG. 8A). Effective diffusivity of Fc-Noggin from the HAKT-gel (5.4×10⁻¹²±1.4×10⁻¹² m²/s) was reduced in the presence of FcL-1 (3.7×10⁻¹²±5.5×10⁻¹³ m²/s) (FIG. 8B). Possible non-specific interactions with the gel may have an impact in Fc-Noggin release. We examined the role of supernatant pH to further investigate this behavior. Fc-Noggin presents a theoretical isoelectric point of 8.9, therefore it would be expected that supernatant pH can alter polar and electrostatic interactions of Fc-Noggin with the delivery matrix as a result of a change in protein charge. HAKT-gel containing Fc-Noggin was subjected to release media at pH 7.4, 9.0, and 10. Protein release was affected only in the first 3 days, with the greatest amount of protein release obtained at pH 10 (FIG. 8C). We then hypothesized that delayed release was caused due to electrostatic interactions, therefore by raising the level of competing ions in solution, adsorption mediated by electrostatic interactions may be disrupted. Addition of 250 mM NaCl into release media supernatant significantly increased the release rate of Fc-Noggin from HAKT-gel (FIG. 8D).

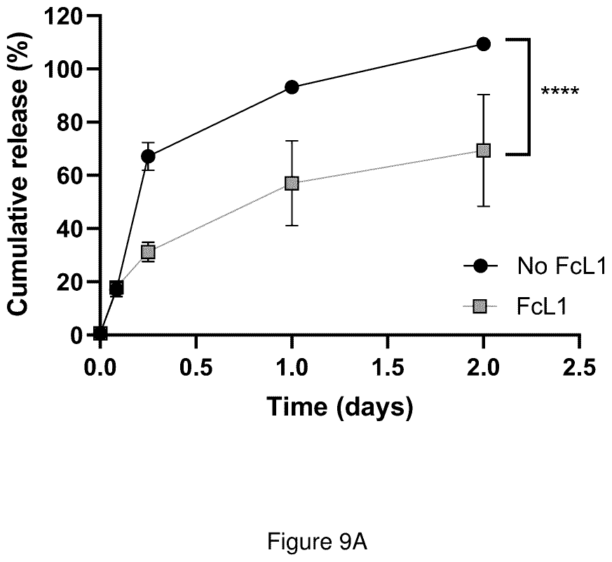

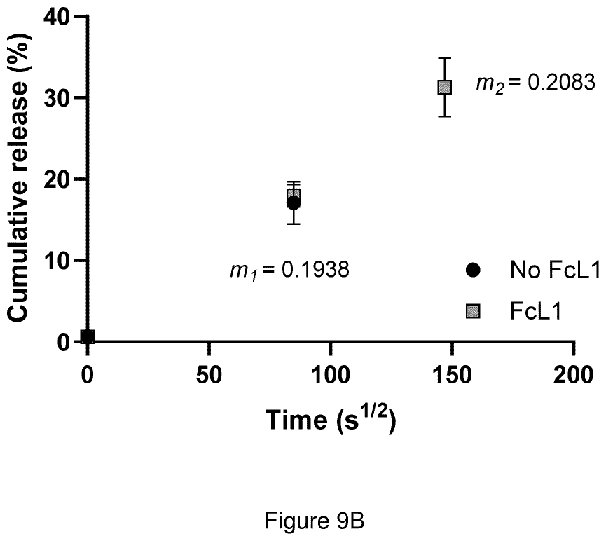

We also confirmed controlled release of bevacizumab in the presence of FcL-1. After 2 days, approximately 100% of bevacizumab was released from HAKT-oxime while only 60% from HAKT-FcL1-oxime (FIG. 9A). Release rates were compared by plotting the cumulative antibody release as a function of the square root of time. The linear region in this plot is indicative of Fickian diffusion, and the slopes of the fit represent the protein diffusivity within the gel. We determined that the relative diffusion coefficients for bevacizumab from HAKT-oxime and HAKT-FcL1-oxime gels were not significantly different; however, HAKT-oxime showed Fickian diffusion release for the first 2 h, while HAKT-FcL1-oxime showed extended Fickian diffusion to 2 days indicating prolonged controlled release (FIG. 9B).

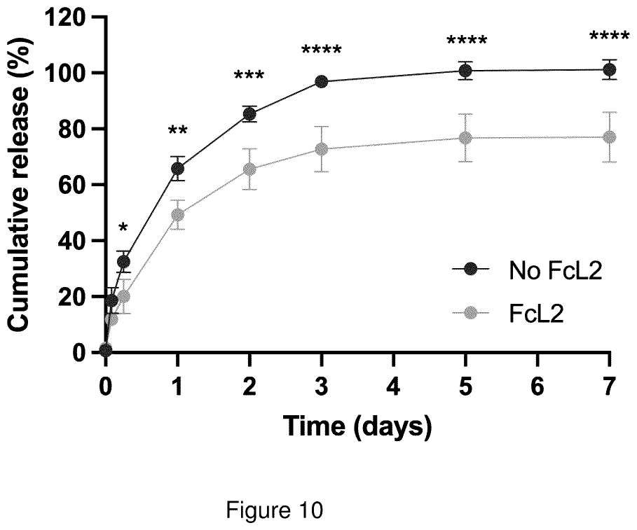

AntisFRP2 release was evaluated in the presence of FcL2 to validate its performance on the controlled release of this antibody. FcL2 was immobilized onto HAKT via IEDDA chemistry, resulting in 11.2 nmol peptide/mg HAKT as determined by the Pierce™ Quantitative Fluorometric Peptide Assay. Anti-sFRP2 was released from HAKT-gel containing no peptide and 100-fold molar excess FcL2 (FIG. 10). Controlled release was achieved in the presence of FcL2, confirming the hypothesis that Fc binding peptides immobilized within a hydrogel delivery vehicle can be leveraged for the controlled release of antibodies. Nevertheless, significant attention needs to be paid to the rate of dissociation (koff) as this is a key parameter in affinity-based controlled release systems (Vulic et al., 2015).

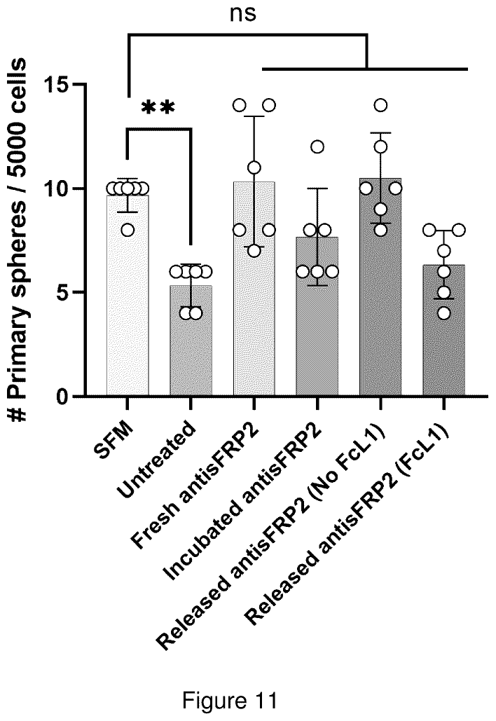

Anti-sFRP2 has been shown to play a significant role in the activation of endogenous retinal stem cells through the inhibition of sFRP2 (Balenci et al., 2013). To investigate the bioactivity of released anti-sFRP2, adult CE-derived cells were cultured in the presence of 10 ng/mL sFRP2 and 50 ng/mL anti-sFRP2. We compared anti-sFRP2 released from the gel containing no FcL1 and 100-fold molar excess FcL1 to antibody to fresh anti-sFRP2 and anti-sFRP2 incubated at 37° C. for 7 days (FIG. 11). The released anti-sFRP2 remained bioactive, presenting similar bioactivity to both fresh anti-sFRP2 and incubated anti-sFRP2, with no significant differences.

### Significance

Polymeric nanoparticles are an attractive approach for the delivery of small molecule drugs; however, their application to the delivery of protein therapeutics is hindered by the use of organic solvents and/or shear forces during formulation that can be detrimental to protein function and structure. Affinity-based systems have emerged as an alternative strategy for the delivery of proteins from a neutral aqueous environment such as hydrogels. Previous work has achieved sustained release of fusion proteins containing a Src homology domain 3 (SH3) through the incorporation of SH3 affinity peptides in a hydrogel vehicle. This strategy works; however, it requires protein modification to achieve controlled release. The skilled person would know that any protein modification never yields 100% modification and the protein modification usually requires additional purification steps leading to a loss in proteins from the starting protein amount. This work demonstrates a general approach to sustaining the release of antibodies or proteins containing the fragment crystallizable domain (Fc) of immunoglobulin G (IgG), IgA, IgD, IgE or IgM.

By incorporating Fc-peptide ligands within a hyaluronan-based hydrogel vehicle we could enable controlled and tunable release without the need for antibody modification. Affinity chromatography is usually used for protein purification. The proteins bind to the antibodies or peptides immobilized on the surface of the chromatography particles. Affinity chromatography aims at the release of the proteins once the proteins are pure, typically within minutes or some hours.

Protein-release systems using hydrogels for the sustained release of proteins aim at a sustained release over hours, days and/or weeks. As opposed to affinity chromatography particles, where the proteins are bound to the surface of the particles, the protein-release systems have the proteins primarily within the hydrogel. Sustained release from the hydrogels may not only be tailored by varying the affinity binding of the proteins and the peptidic ligands but also controlling the degree of crosslinking and the mesh size of the polymeric network. Interactions, such as electrostatic and hydrophobic, between the polymeric backbone and the proteins may also influence the release rate of the proteins from the hydrogel. This platform could be applied to a wide range of antibodies or Fc-fusion proteins.

### References

Baker, A. E. G., Cui, H., Ballios, B. G., Ing, S., Yan, P., Wolfer, J., Wright, T., Dang, M., Gan, N. Y., Cooke, M. J., Ortin-martinez, A., Wallace, V. A., Kooy, D. Van Der, Devenyi, R., & Shoichet, M. S. (2021). Stable oxime-crosslinked hyaluronan-based hydrogel as a biomimetic vitreous substitute. Biomaterials, 271 (February), 120750. https://doi.org/10.1016/j.biomaterials.2021.120750
Balenci, L., Wonders, C., Coles, B. L. K., Clarke, L., & Van Der Kooy, D. (2013). Bone morphogenetic proteins and secreted frizzled related protein 2 maintain the quiescence of adult mammalian retinal stem cells. Stem Cells, 31(10), 2218-2230. https://doi.org/10.1002/stem.1470
Hettiaratchi, M. H., Krishnan, L., Rouse, T., Chou, C., McDevitt, T. C., & Guldberg, R. E. (2020). Heparin-mediated delivery of bone morphogenetic protein-2 improves spatial localization of bone regeneration. In Science Advances (Vol. 6, Issue 1). https://doi.org/10.1126/sciadv.aay1240
Hettiaratchi, M. H., & Shoichet, M. S. (2019). Modulated Protein Delivery to Engineer Tissue Repair. In Tissue Engineering—Part A (Vol. 25, Issues 13-14, pp. 925-930). Mary Ann Liebert Inc. https://doi.org/10.1089/ten.tea.2019.0066
J, B., R S, P., D, X., Y H, L., H, S., Q, W., & J, Y. (2015). Intravitreal anti-VEGF injections for treating wet age-related macular degeneration: a systematic review and meta-analysis. Drug Design, Development and Therapy, 9, 5397-5405. https://doi.org/10.2147/DDDT.S86269
Lu, R.-M., Hwang, Y.-C., Liu, I.-J., Lee, C.-C., Tsai, H.-Z., Li, H.-J., & Wu, H.-C. (2020). Development of therapeutic antibodies for the treatment of diseases. Journal of Biomedical Science 2020 27:1, 27(1), 1-30. https://doi.org/10.1186/S12929-019-0592-Z
Meyer, C. H., & Holz, F. G. (2011). Preclinical aspects of anti-VEGF agents for the treatment of wet AMD: ranibizumab and bevacizumab. Eye 2011 25:6, 25(6), 661-672. https://doi.org/10.1038/eye.2011.66
Mullard, A. (2021). FDA approves 100th monoclonal antibody product. Nature Reviews. Drug Discovery, 20(7), 491-495. https://doi.org/10.1038/D41573-021-00079-7
Pakulska, M. M., Donaghue, I. E., Obermeyer, J. M., Tuladhar, A., McLaughlin, C. K., Shendruk, T. N., & Shoichet, M. S. (2016). Encapsulation-free controlled release: Electrostatic adsorption eliminates the need for protein encapsulation in PLGA nanoparticles. Science Advances, 2(5). https://doi.org/10.1126/sciadv.1600519
Reese, H. R., Shanahan, C. C., Lembo, J., Tsonev, L., Hirsh, A., & Menegatti, S. (2020). Chromatographic assay to probe the binding energy and mechanisms of homologous proteins to surface-bound ligands. Journal of Chromatography B, 1136, 121927. https://doi.org/10.1016/J.JCHROMB.2019.121927
Verdoliva, A., Marasco, D., de Capua, A., Saporito, A., Bellofiore, P., Manfredi, V., Fattorusso, R., Pedone, C., & Ruvo, M. (2005). A new ligand for immunoglobulin G subdomains by screening of a synthetic peptide library. ChemBioChem, 6(7), 1242-1253. https://doi.org/10.1002/cbic.200400368
Vulic, K., Pakulska, M. M., Sonthalia, R., Ramachandran, A., & Shoichet, M. S. (2015). Mathematical model accurately predicts protein release from an affinity-based delivery system. Journal of Controlled Release, 197, 69-77. https://doi.org/10.1016/J.JCONREL.2014.10.032
Wykoff, C. C., Clark, W. L., Nielsen, J. S., Brill, J. v., Greene, L. S., & Heggen, C. L. (2018). Optimizing Anti-VEGF Treatment Outcomes for Patients with Neovascular Age-Related Macular Degeneration. Https://Doi.org/10.18553/Jmcp.2018.24.2-a.S3, 24(2a), S3—S15. https://doi.org/10.18553/JMCP.2018.24.2-A.S3
Yang, H., Gurgel, P. V., Williams, D. K., Jr, Bobay, B. G., Cavanagh, J., Muddiman, D. C., & Carbonell, R. G. (2010). Binding site on human immunoglobulin G for the affinity ligand HWRGWV. Journal of Molecular Recognition : JMR, 23(3), 271. https://doi.org/10.1002/JMR.967

Therefore what is claimed is:

1. A protein-release system for sustained release of proteins, comprising:
a hydrogel comprising a polymeric network;
proteins having a portion corresponding to a portion of a fragment crystallization (Fc) constant region of an antibody; and
peptidic ligands covalently coupled to the polymeric network, the peptidic ligands comprising an amino acid sequence having a binding affinity to the portion of the Fc constant region, each protein being reversibly bound by affinity to the peptidic ligands, wherein the polymeric network comprises naturally-derived polymer, synthetic polymer or a combination thereof.

2. The protein-release system according to claim 1, wherein the binding affinity is characterized by a dissociation constant between the protein constant region and the peptidic ligand greater than about 10⁻¹⁰ M.

3. The protein-release system according to claim 1, wherein the dissociation constant is in a range from about 10⁻⁴ to about 10⁻⁹ M.

4. The protein-release system according to claim 1, wherein the peptide sequence has an amino acid sequence selected to give a preselected dissociation constant to give a selected release rate of the protein.

5. The protein-release system according to claim 1, wherein the proteins are any one or combination of IgG, IgA, IgD, IgE or IgM antibodies or fusion proteins comprising a portion of a Fc region of an IgG, IgA, IgD, IgE or IgM antibody.

6. The protein-release system according to claim 1, wherein the peptidic ligand comprises a chain extender.

7. The protein-release system according to claim 1, wherein the amino acid sequence comprises the sequence selected from the group consisting of HWRGWV, HYFKFD, HFRRHL, HWCitGWV, HWRGWVKGKASKG, (CFHH)₂KG, NVQYFAV, FYWHCLDE, FYTHCAKE, FYCHTIDE, GSYWYQVWF, RRGW and DCAWHLGELVWCT.

8. The protein-release system according to claim 1, wherein the proteins are a mixture of two or more different proteins.

9. The protein-release system according to claim 1, wherein the peptidic ligands are a mixture of two or more ligands having a different amino acid sequence.

10. A method of producing a protein-release system for sustained release of proteins, the proteins having a portion corresponding to a portion of a Fc constant region of an antibody, the method comprising:
a) selecting a peptidic ligands known to have a binding affinity with the Fc region;
b) covalently immobilizing the peptidic ligands to polymeric network precursors;
c) mixing aqueous solution of polymeric network precursors comprising the immobilized peptidic ligands with the proteins; and
d) forming a hydrogel loaded with proteins for sustained release wherein the polymeric network comprises naturally-derived polymer, synthetic polymer or a combination thereof.

11. The method according to claim 10, wherein the hydrogel is formed in situ

12. The method according to claim 10, wherein the formed hydrogel is formed as nanoparticles or microparticles.

13. The method according to claim 10, wherein the binding affinity is characterized by a dissociation constant between the protein constant region and the peptidic ligand greater than about 10⁻¹⁰ M.

14. The method according to claim 10, wherein the peptide sequence has an amino acid sequence selected to give a preselected dissociation constant to give a selected release rate of the protein.

15. The method according to claim 10, wherein the proteins are any one or combination of IgG, IgA, IgD, IgE or IgM antibodies or fusion proteins comprising a portion of a Fc region of an IgG, IgA, IgD, IgE or IgM antibody.

16. The method according to claim 10, wherein the peptidic ligand comprises a chain extender.

17. The method according to claim 10, wherein the amino acid sequence comprises the sequence selected from the group consisting of HWRGWV, HYFKFD, HFRRHL, HWCitGWV, HWRGWVKGKASKG, (CFHH)₂KG, NVQYFAV, FYWHCLDE, FYTHCAKE, FYCHTIDE, GSYWYQVWF, RRGW and DCAWHLGELVWCT.

18. The method according to claim 10, wherein the proteins are a mixture of two or more different proteins, each protein having a selected binding affinity with the peptidic ligands.

19. The method according to claim 10, wherein the proteins are a mixture of two or more different proteins and the peptidic ligands are a mixture of two or more different peptidic ligands, each protein having a selected binding affinity with each peptidic ligands.

20. The method according to claim 10, wherein the peptidic ligands are a mixture of two or more ligands having a different amino acid sequence.
